(19) United States
(12) Patent Application Publication
(10) Pub. No.: US 2026/0041367 A1
(43) Pub. Date: Feb. 12, 2026

(54) SYSTEM AND METHOD FOR DETECTING MOTION DISORDER OF A USER OF A HEAD-WEARABLE DEVICE

(71) Applicant: Sivantos Pte. Ltd., Singapore (SG)

(72) Inventors: Michael BLENDINGER, Erlangen (DE); Julius KRIEG, Olpe (DE)

(21) Appl. No.: 18/800,310

(22) Filed: Aug. 12, 2024

(51) Int. Cl. A61B 5/00; A61B 5/05; H04R 25/00

(52) U.S. Cl. CPC A61B 5/4082; A61B 5/05; A61B 5/6803; A61B 5/7264; A61B 5/746; H04R 25/609; A61B 2562/0219; A61B 2562/0223

(57) ABSTRACT

A system for detecting motion disorder of a user of a head-wearable device includes: at least one motion sensor, providing motion data of the user of the head-wearable device; the head wearable device, with a processing unit for processing the motion data provided by the at least motion sensor and with a memory unit for storing the processed motion data. The processing unit has a processor adapted to perform the following: i) analyzing the motion data and thereby generating analyzed motion data, including classifying the motion data to different motion classes; j) storing the analyzed motion data; k) comparing current motion data with reference motion data, wherein the reference motion data is selected from the group of previously stored motion data of the same motion class and external reference motion data; and l) submitting a warning signal if the current motion data inadmissible differs from the reference motion data.

### BACKGROUND OF THE INVENTION

### Field Of The Invention

The invention relates to a System and a method for detecting motion disorder of a person with the help of a head-wearable device. The invention relates especially to detect prodromal symptoms of motion disorder of the wearer (user) of the head-wearable device, in particular neurodegenerative disorders such as Parkinson's.

### Description of the Background Art

Said disorders typically start affecting the behavior of future patients in their everyday life months or years before they become aware of the problem and seek medical attention to obtain a diagnosis.

Such neurodegenerative disorders, especially diseases belonging to the ‘parkinsonian group of disorders, effect the functions of the basal ganglia in movement control. The basal ganglia plays a complex role in the modulation of motor behavior. Basal ganglia function may also be deteriorated for other reasons, examples comprise tumor diseases or chronic fatigue syndrome. As a result, movement patterns can be used as an indicator of basal ganglia function and, therefore as an indicator for the presence of a motion disorder.

For the diagnosis of Parkinson's or similar motion disorder it is well known in the prior art to stimulate the person and to measure the motion-reaction with respect to this external stimulation.

According to US 2022/0361787 A1 a system is described with an ear-worn device, which is configured to initiate a generation of a stimulus sufficient to generate a response from the ear-worn device wearer. The ear-worn device is further configured to monitor a qualified response of the wearer to the stimulus.

### SUMMARY OF THE INVENTION

An object of the invention is to provide an improved system and an improved method for detecting motion disorder of a user (wearer) of a head-wearable device, especially it is an object to detect prodromal symptoms of motion disorder of the user, in particular neurodegenerative disorders such as Parkinson's.

The inventive system is designed for detecting motion disorder of a user (wearer) of a head-wearable device, the system comprising:
at least one motion sensor, providing motion data of the user of the head-wearable device, wherein the motion data preferably comprising a motion signal recorded by the motion sensor,
the head wearable device, which comprises a processing unit, especially a digital signal processing unit for processing the motion data provided by the at least one motion sensor and further comprising a memory unit for storage of the motion data processed by the processing unit as processed data,
said processing unit having a processor, for example a microchip, which is adapted to perform the following steps:
   a) analyzing the motion data and thereby generating analyzed motion data, the step of analyzing comprising classifying the motion data to different motion classes,
   b) storing the analyzed motion data,
   c) comparing current motion data with reference motion data, wherein the reference motion data is selected from the group of previously stored motion data of the same motion class and external reference motion data, wherein the previously stored motion data are previously analyzed motion data and wherein the external reference motion data are taken especially from a lock up table, preferably stored in the memory,
   d) submitting a warning signal if the current motion data inadmissible differs from the reference motion data, for example the current motion data deviates by a specified tolerance range from the reference motion data

The inventive method for detecting motion disorder of a user of a head-wearable device comprising the following steps
providing motion data of the user of the head-wearable device with the help of at least one motion sensor wherein the at least one motion sensor is preferably part of the head-wearable device and wherein the motion data preferably comprising a motion signal recorded by the at least motion sensor,
processing the motion data provided by the at least motion sensor and storing of the motion data processed by the processing unit in a memory unit, as processed data
the processing of the motion data comprising the following steps:
   e) analyzing the motion data and thereby generating analyzed motion data, the step of analyzing comprising classifying the motion data to different motion classes,
   f) storing the analyzed motion data,
   g) comparing current motion data with reference motion data, wherein the reference motion data is selected from the group of previously stored motion data of the same motion class and external reference motion data,
   h) submitting a warning signal if the current motion data inadmissible differs from the reference motion data.

The advantages and preferred embodiments mentioned below with regard to the system can also be applied analogously to the method and vice versa.

The external reference motion data might be public available data, for example from scientific studies/publications.

The collected and recorded current motion data is for example continuously compared or periodically compared to the reference motion data.

If the reference motion data is external reference motion data, the reference data is preferably derived from people (reference group) with similar or the same physiological characteristics like the user. Such physiological characteristics can be age, sex, and additionally weight and height of the person, for example.

If there is a certain divergence between the current motion data and the reference motion data, the user of the head wearable device will be informed by the warning signal that there is an indication of a possible motion disorder and check by a doctor should be considered. Therefore, the result of the comparison is a motion health measure. If this determined motion health measure is outside of a predefined limit, the notification in form of the warning signal is provided. Due to the storing of the motion data, valuable data is collected with a head wearable device, which therefore is a wearable health platform to support the retrospective data analysis and to support a doctor making a diagnosis.

Such a warning signal might be an acoustic or an optic signal, e.g. displayed or generated by the head-wearable device or another device like a handheld device like a smartphone, which is connected with the head-wearable device for example via a (wireless) connection link like Bluetooth. Also, the warning signal might be an electronic signal transmitted to a different device like a computer, for example via the internet. In such cases the head wearable device has a communication interface for communication with the external device.

Due to the classification only comparable movements of the user are compared with each other. For example, at least some of the following classes of movements are used:
movement of the head
movement of limbs
movement of the body
walking (fast/slow)
climbing stairs
and so on . . .

The invention is based on the knowledge that later Parkinson diagnosed patients already show a reduced motion behavior, especially a reduced acceleration well before the diagnosis of Parkinson. Such a reduced motion behavior is detected by the System and the method in an early stage and can detect such prodromal symptoms.

According to a preferred embodiment a baseline is extracted based on a number of different analyzed motion data of the same class, the baseline defining the reference motion data with which the current motion data is compared. The baseline might be a mean value of the different motion data or other statistic evaluations of the different stored motion data.

The baseline therefore defines a former (reference) behavior of the specific user such that the current motion data can compared with such former behavior. The baseline is preferably based only on such previously collected motion data which have been recorded before a specified period of time e.g. several months or at least a year.

Preferably, a plurality of baselines is generated, which refer to different time periods. That means that the different baselines have a different age and only cover specific time periods of the recorded motion data.

Therefore, the current motion data can be compared to different baselines. These different baselines define the reference motion data.

Preferably based on a number of current motion data (e.g. over a specified time period of for example one day or one week or even one month) a current baseline is generated. Also, the current baseline might be a mean value of the different current motion data or other statistic evaluations of the different current motion data. This current baseline defines the current motion data which is compared with the reference motion data.

In general, current motion data is motion data is preferably recorded by the motion sensor in the last few days or the last (few weeks). In every case the reference data is much older (more than month or at least one year) than the current motion data.

In a preferred embodiment the processer is adapted to analyze transitions between motions, wherein the transition is stored as the analyzed motion data.

Under transition between motions it is understood to move a part of the body from an initial position to a second position, like
turning the head from one direction to a different direction
raising/lowering a limb, like an arm/leg.
. . .

Under transition between motions, it is furthermore understood a transition between different kinds or types of motions, the different kinds might be classified by
activity (no activity—starting movement, e.g. sitting-walking)
complexity (low complexity—high complexity, e.g. movement only of one part of the body compared to coordinated movement of different parts of the body)
acceleration (low acceleration—high acceleration of the movement)

The analyzing of such transitions is an essential concept of the present invention. The transition behavior allows to detect the prodromal symptoms of motion disorder. Especially the analyzing of such transitions allows to detect a different (delayed or surprisingly) initiation of a movement, compared a normal behavior.

Therefore, with the invention signs of delayed or suppressed initiation of movement are detected as a biomarker for neurodegenerative disease.

Preferably the analyzed current transition behavior defines the current motion data which is compare to a reference transition behavior. The reference transition behavior might be derived from previously recorded motion data or alternatively from external reference data like already explained above.

The transitions are analyzed especially in view of
a time delay between the initial and the following movement and/or
velocity and/or acceleration of the following movement

According to a first concept, only the transitions are analyzed and used for detecting the motion disorder of the user.

According to a preferred embodiment the duration of an initial motion is detected, wherein the initial motion is followed by a following motion, and wherein the duration is a parameter of the transition behavior. Each transition between motions defines in general a transition from an initial motion (which even might be a non-motion) to a second, following motion. The evaluation of the duration of the initial motion provides additional insights in the transition behavior and especially allows the evaluation of an urge for motion because a longer duration of the first activity (initial motion) may be related to an inhibited inclination to change to the following motion.

Preferably the motion data is classified especially according to a complexity of the motion, wherein for classifying the motion data and especially the complexity, a motion signal, provided by the motion sensor is analyzed with the help of a signal analyzer.

Especially the signal is analyzed in view of its frequency behavior, and/or the course of the signal level, the periodicity and so on.

Preferable one or more of the following parameters of the motion data, especially of the motion signal are analyzed:
spectral behavior, especially a spectral flatness
eigenvalue spread
Lempel-Ziv complexity
Entropy
characteristics of a periodogram
an autoregressive estimation
or a combination or a variation of the mentioned parameters.

Based on these analyses the current movement is classified in one of the different classes as they have been mentioned above, for example.

Preferably, the at least one motion sensor is selected from at least one of the following sensors:
an accelerometer
a gyroscope
a magnetometer
or a combination or variation thereof.

Especially an accelerometer is used to measure an acceleration of movements, especially measuring the acceleration of a transition, that is for example the acceleration of the second movement which is following the initial movement.

Preferably, the at least one motion sensor is part of the head wearable device. Besides that integrated motion sensor other external sensors might also be used. However preferable, only motion sensors integrated in the head wearable device are used.

With such an integrated sensor different movements of the users can be detected and monitored. This is already known. Besides the movements of the head also movements of the body and or limbs of the user can be detected and analyzed, especially based on the movements of the head.

Preferably, movements which are stimulated by an external and therefore by an environmental stimulation are not taken into account and therefore are not considered. Such external/environmental stimulations are for example external sound, an external action of other people or any other external/environmental happenings/actions (not initiated by the user).

Often the diagnosis or evaluation of a motion disorder like Parkinson is based on such external stimulations and the measurement of the reaction of the user. In opposite to this, the present invention dispenses with such external stimulation.

Examples for such external stimulations are foreign voice activity (e.g., somebody calls the user's name or speaks to him), environmental sounds (e.g., a slamming door, a door bell, operation sounds of household devices, traffic, environmental noise, and so on).

Therefore, the movements which are analyzed are based only on intrinsic movements of the user without any external stimulation. Only movements are considered, which are initiated by the user itself without external stimulation.

Preferably, the wearable device comprises a stimulation sensor, which is able to detect the external stimulation. Preferably the signal of the stimulation sensor is analyzed regarding the occurrence of such an external stimulation Preferably the stimulation sensor sends a signal for example to the to the processing unit for analyzing the stimulation signal.

If it is recognized that an external stimulation has occurred, the motion data of the motion sensor preferably is not considered for the analyses, if there is a motion disorder. Especially the processor is adapted to decide based on the signals presented by the stimulation sensor, if an external stimulation has occurred. This assures that the presence of an external stimulus is detected and that only stimulation-free motion data is stored as analyzed data.

Preferably, if it is recognized that an external stimulation has occurred, the motion data of the motion sensor is analyzed to determine if there has been a reaction of the user. This assessment is preferably done by the processing unit.

This measure is based on the consideration that for example a user's failure to react to an external stimulus or a delayed reaction of the user to an external stimulation might also be a prodromal symptom for a motion disorder. Especially a long-term surveillance of a plurality of such failures are stored and analyzed. Long-term means in this case the surveillance over multiple months or even at least one year.

Preferably the motion data are analyzed in view of a reaction time of the user between the external stimulation and the reaction of the user (initiating a movement). This reaction time defines a time delay between the occurrence of the external stimulus and the transition from the first, initial motion to the following second motion. This delay is stored as the analyzed motion data.

For the decision, if an external stimulation occurred, preferably the signals of the stimulation sensor are classified for example based on signal level, especially a sound level. If the signal level exceeds for example a defined threshold this is judged as occurrence of an external stimulation.

Preferably the stimulation sensor is part of the head-wearable device. And especially the stimulation sensor is a microphone. If the head wearable device is for example a hearing device, especially a hearing aid with an integrated microphone, such an already existing microphone is used as the stimulation sensor.

Alternatively other stimulation sensors might be used which are connected (especially wirelessly connected) to the head wearable device. Such another stimulation sensor might be integrated for example in a handheld device like a smartphone or it might be such a handheld device. The microphone of such a handheld device might be used. Alternatively, notices provided by the handheld device might be used as stimulation signal and the notices which might be judged as an external stimulation, for example an incoming message or phone call.

Preferably, the motion data is recorded, analyzed and stored over a long term of at least weeks, months and even years. Therefore, a long-term monitoring of the user's motion behavior is performed.

Preferably, only conscious movements of the user are detected and considered for the judgment and for the analyze, if there might be a motion disorder. Uncontrolled or unintended movements, like tremor are not considered.

Preferably, no gait analyses are done. Therefore, the gait of the user is not analyzed for the determination if there is a motion disorder.

Preferably, the head wearable device is a hearing aid for hearing impaired people. Such hearing aids have the main advantage that they are worn normally the whole day and that they are therefore suitable for the monitoring the movements of the user in his everyday life the whole day. Such hearing aids are personalised in such a way that specific, user dependent fittings are made to overcome user specific hearing deficiencies as this is well known in the technical field of hearing aids.

Alternatively, the head wearable device is a headphone or earphone which is used to listen to music, for example.

With the present system and method prodromal symptoms of motion disorder might be detected such that an early diagnosis and treatment may at least delay the progression of the disease. Another benefit of a regularly worn system, like a hearing aid that performs such medical evaluations as described above, is the calming feeling for the users. They are aware that all possible analyses are performed with their data to ensure their health and wellbeing.

### BRIEF DESCRIPTION OF THE FIGURES

An example of the invention is described with reference to the figures. These figures show in simplified illustrations

FIG. 1 a schematic representation of a hearing aid as a head-wearable device,

FIG. 2 an illustration of the classification process and storing of the analyzed motion data.

### DETAILED DESCRIPTION

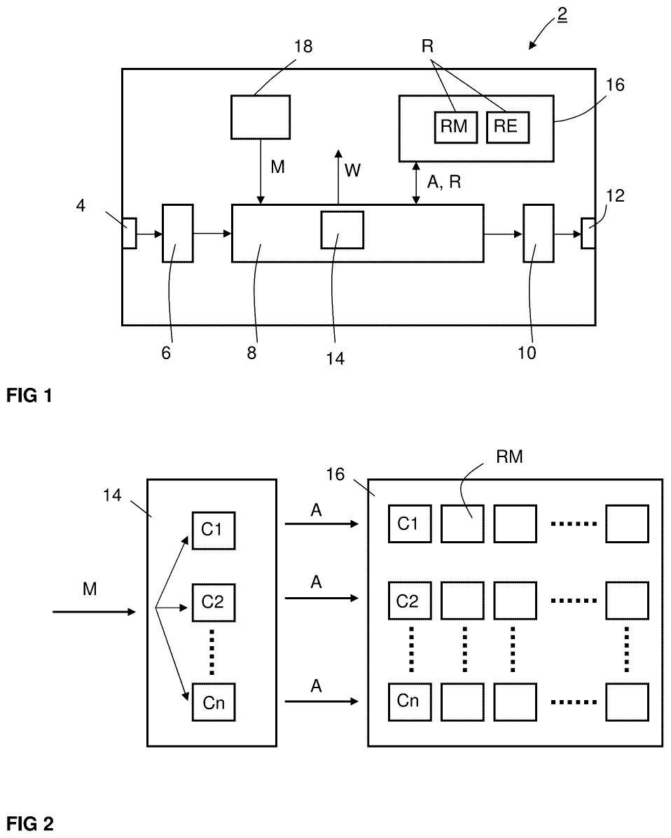

The hearing aid 2 shown in FIG. 1 is designed to support a hearing-impaired person, called user in the following. There are different kind of hearing aids know, for example BTE (Behind The Ear), ITE (In The Ear), ITC (In The Canal) hearing aid.

The hearing aid 2 generally has a housing or shell, which houses the different parts of the hearing aid. In FIG. 1 only some of these parts are shown:

The hearing aid 2 comprises normally at least one input receiver, especially a microphone 4, an A/D signal converter 6 for converting an acoustic input signal of the microphone 4 to a digital input signal, a digital processing unit 8 for processing the digital input signal and to output a processed digital output signal.

This digital output signal is normally converted to an analogue output signal with the help of a D/A signal converter 10 and forwarded to an output receiver, especially an acoustic loudspeaker 12.

The digital processing unit 8 has a processor 14, for example a microchip. For the normal operation of the hearing aid 2, that means to assist the hearing-impaired user and to compensate the user specific hearing defencies, the digital input signal is processed by the digital processing unit 8 based und user specific fitting parameters.

The functions and the operation of the hearing aid for compensating the hearing loss of the user is well known and therefore not described in detail.

Furthermore, the hearing aid 2 comprises a memory unit 16 and additionally a motion sensor 18, especially an acceleration sensor.

When the user is wearing the hearing aid, the motion sensor 18 provides motion data M, especially in the form of motion signals, e.g. digital signals. This motion data M is sent to the digital processing unit 8 and is processed and analyzed. The processed motion data M is outputted as analyzed data A and stored in the memory unit 16.

During the processing of the current motion data M these motion data are classified in different classes C1 . . . Cn as shown in FIG. 2.

This process is done continuously or periodically such that in the course of time a plurality of classified analyzed data A of different age is received.

These classified analyzed data defines stored motion data RM which defines reference motion data R.

Especially, based on different analyzed data A of the same class C1 . . . Cn, for example by averaging of different analyzed data A of the same class C1 . . . Cn, at least one processed motion data is extracted, which is called a (reference) baseline. It is possible to extract and store different baselines for example of different age. Each of these baselines defines stored motion data RM

This stored motion data RM is stored in the memory unit 16 (pls. compare FIG. 1).

Additionally or as an alternative, external reference motion data RE is stored in the memory unit 16, for example as a look-up table.

The processing unit 8 and especially the processor is designed in such a way to analyze the motion data M for the assessment if there might be a motion disorder of the user.

For this, the processing unit compares the current analyzed motion data A with the reference motion data R, for example with the stored motion data RM or with the external reference motion data.

If the current analyzed motion data A (which might even be derived from a plurality of analyzed motion data A, for example by averaging) differs from the reference motion data more than a predefined threshold, the processing unit 8 outputs a warning signal W, especially to the user. This warning signal W can be an acoustic message to the user, provided via the hearing aid 2 itself. Alternatively, the warning signal W is outputted to an external device like a smartphone (not shown).

1. A system for detecting motion disorder of a user of a head-wearable device comprising:
at least one motion sensor, providing motion data of the user of the head-wearable device,
the head wearable device comprising a processing unit for processing the motion data provided by the at least one motion sensor, a memory for storage of the motion data processed by the processing unit, and a microphone forming a stimulation sensor that is configured to detect an external stimulation;
said processing unit having a processor, which is adapted to perform the following steps:
a) analyzing the motion data and thereby generating analyzed motion data, the step of analyzing comprising classifying the motion data to different motion classes,
b) storing the analyzed motion data,
c) comparing analyzed motion data with reference motion data, wherein movements that are stimulated by an external stimulation are disregarded and not taken into account, and wherein the reference motion data is selected from a group of previously stored analyzed motion data of the same motion class and external reference motion data,
d) submitting a warning signal if the analyzed motion data of movements that are taken into account differs from the reference motion data, and
wherein said processor is adapted to decide, based on signals presented by said stimulation sensor, whether an external stimulation has occurred, and if so, to disregard and not take into account the motion data provided by said motion sensor.

2. The system according to the claim 1, wherein based on a number of different analyzed motion data a baseline is extracted, wherein the baseline represents the reference motion data with which the analyzed motion data is compared.

3. The system according to the claim 1, wherein the processor is adapted to analyze transitions between motions, wherein a transition behavior representing the analyzed transitions is stored as the analyzed motion data.

4. The system according to the previous claim claim 3, wherein in step c) the current transition behavior is compared with a reference transition behavior.

5. The system according to claim 4, wherein a duration of an initial motion is detected, wherein the initial motion is followed by a following motion, and wherein the duration is a parameter of the transition behavior.

6. The system according to claim 1, wherein in step a) for classifying the motion data a motion signal provided by the motion sensor is analyzed with the help of signal analyses.

7. The system according to claim 1, wherein the at least one motion sensor is se-lected from at least one of the following sensors:
a. an accelerometer
b. a gyroscope
c. a magnetometer.

8. The system according to claim 1, wherein the motion sensor is part of the head wearable device.

9-11. (canceled)

12. The system according to claim 1, wherein the processor is adapted to decide based on the signals presented by the stimulation sensor, if an external stimulation has occurred, and if so, the motion data provided by the motion sensor is analyzed to determine if there has been a reaction of the user.

13. The system according to claim 12, wherein the motion data is analyzed to determine a delayed reaction of the user with respect to the stimulation.

14. (canceled)

15. The system according to claim 1, wherein the motion data is recorded, analyzed and stored over a long term of at least one of weeks, months and years.

16. The system according to claim 1, wherein the processor is adapted to detect only conscious movements of the user and not uncontrolled movements

17. The system according to claim 1, wherein the processor does not carry out any gait analyses.

18. The system according to claim 1, wherein the head wearable device is a hearing aid for hearing impaired people.

19. A method for detecting motion disorder of a user of a head-wearable device, the method comprising the steps
providing motion data of the user of the head-wearable device with the help of at least one motion sensor,
processing the motion data provided by the at least one motion sensor and storing of the motion data processed by the processing unit in a memory unit,
the processing of the motion data comprising the following steps:
e) analyzing the motion data and thereby generating analyzed motion data, and not taking into account movements that are stimulated by an external stimulation,
f) storing the analyzed motion data,
g) comparing the analyzed motion data with reference motion data, wherein the reference motion data is selected from the group of previously stored motion data of the same motion class and external reference motion data,
h) submitting a warning signal if the analyzed motion data of movements that are taken into account differs from the reference motion data; and
deciding, based on signals presented by the stimulation sensor, whether an external stimulation has occurred, and if so, to disregard and not take into account corresponding motion data provided by the motion sensor.
